(19) United States
(12) Patent Application Publication
(10) Pub. No.: US 2025/0058103 A1
(43) Pub. Date: Feb. 20, 2025

(54) METHOD FOR ARBITRATION BETWEEN PULSES IN A NEUROSTIMULATION DEVICE

(71) Applicant: Boston Scientific Neuromodulation Corporation, Valencia, CA (US)

(72) Inventor: Philip Leonard Weiss, Sherman Oaks, CA (US)

(73) Assignee: Boston Scientific Neuromodulation Corporation, Valencia, CA (US)

(21) Appl. No.: 18/802,762

(22) Filed: Aug. 13, 2024

Related U.S. Application Data
(60) Provisional application No. 63/532,620, filed on Aug. 14, 2023.

(51) Int. Cl. A61N 1/05; A61N 1/36

(52) U.S. Cl. CPC A61N 1/0534; A61N 1/36125; A61N 1/36175

(57) ABSTRACT

Methods and devices for controlling arbitration in an implantable medical device capable of issuing therapy stimulus for a plurality of therapy programs. The system defines a therapy program at multiple levels including pulse programs, aggregate instructions, and therapy programs. Holdoff and/or arbitration settings are available at several levels of the therapy program definition, allowing increased flexibility and control.

### CROSS REFERENCE TO RELATED APPLICATIONS

The present application claims the benefit of U.S. Provisional Patent Application No. 63/532,620, filed Aug. 14, 2023, titled METHOD FOR ARBITRATION BETWEEN PULSES IN A NEUROSTIMULATION DEVICE, the disclosure of which is incorporated herein by reference.

### BACKGROUND

Arbitration logic and/or instructions are used in implantable medical devices to determine the order of execution of multiple therapy programs. For example, US PG Pub. No. 2013/0184794 describes output circuitry for use in neuromodulation devices, such as spinal cord stimulation (SCS) or deep brain stimulation (DBS) devices. The devices may be programmed to issue multiple therapy programs to different or overlapping sets of therapy electrodes. With multiple therapy programs scheduled to issue therapy outputs using the overlapping electrodes and/or output circuitry, timing controls are used so that contrary instructions do not reach the same output-defining circuits at the same time. For example, without arbitration, a single output digital-to-analog converter (DAC) could be commanded to issue a positive one milliamp current at the same time as it is commanded to issue a negative two milliamp current, causing problems with both of the competing therapy programs. Prior systems have used arbitration in a manner which is sometimes not granular enough to allow precise control over therapy programs. As more sophisticated therapy programs are desired with current and future systems, new and/or alternative arbitration schemes are desired.

### Overview

The present inventors have recognized, among other things, that a problem to be solved is the need for new and/or alternative arbitration schemes are desired. In illustrative examples, a system for therapy definition is used in which therapy phases are defined at a granular level by pulse instructions, grouped together as aggregate instructions which pair pulse instructions with electrode steering instructions, and the aggregate instructions are further grouped together by therapy configurations. Methods and devices for implementing arbitration and/or pulse holdoff timing at the level of pulse instructions are disclosed. Methods and devices for implementing arbitration and/or pulse holdoff timing at the level of aggregate instructions are also disclosed. Finally, methods and devices for implementing arbitration and/or pulse holdoff timing at the level of therapy configurations are also disclosed.

A first illustrative and non-limiting example takes the form of an implantable medical device comprising: a housing containing a power source, a controller, and stimulation circuitry; and a lead having a plurality of electrodes thereon, the lead coupled to the housing such that the stimulation circuitry can issue stimulus pulse patterns to a patient via the electrodes; wherein the controller comprises: a memory including steering memory, aggregate memory, pulse memory, and configuration memory; a plurality of pulse definition circuits each including steering logic, aggregate logic, and pulse logic; wherein the steering memory contains steering instruction sets for a plurality of steering programs, each steering program determining which of the electrodes receive a fraction of a total stimulus output, and the steering logic is configured to implement a selected steering instruction set; wherein the pulse memory contains pulse programs, each having a one or more pulse instructions defining pulse components each having a pulse type and one or more determining characteristics for the pulse type; wherein the aggregate memory contains aggregate instructions each defining one or more aggregated outputs, each aggregated output pairing a selected steering instruction set with a selected pulse program and defining a number of repetitions for the selected pulse program to execute with the selected steering instruction set; wherein the configuration memory contains a plurality of therapy configuration instruction sets each having a defined total stimulus output amplitude, an arbitration mode, a holdoff setting, and identifying a one or more aggregate instructions to be executed for each therapy configuration; further wherein the arbitration mode defined in the configuration memory for each therapy configuration instruction set determines whether the therapy configuration instruction sets will wait for completion of portions of other therapy configuration instruction sets before initiating, and the holdoff setting determines whether the therapy configuration instruction set can be interrupted by another therapy configuration instruction set; and wherein the controller is configured to execute the plurality of therapy configurations to generate output pulses using the stimulation circuitry by: initiating execution of a first therapy configuration instruction set; while executing the first therapy configuration instruction set, receiving a request to execute a second therapy configuration instruction set; determining whether the arbitration mode for the second therapy configuration instruction set allows the second therapy configuration instruction set to wait for completion of portions of other therapy configuration instruction sets and, if not, initiating execution of the second therapy configuration instruction set while the first therapy configuration instruction set is executing, or else: determining whether the holdoff setting for the first therapy configuration instruction set allows interruption of the first therapy configuration instruction set by the second therapy configuration instruction set and: if so, completing an ongoing execution of at least a portion of the first therapy configuration instruction set, and then starting execution of the second therapy configuration instruction set; or if not, completing execution of the first therapy configuration instruction set before allowing the second therapy configuration instruction set to be started.

Additionally or alternatively, if the holdoff setting for the first therapy configuration instruction set allows interruption of the first therapy configuration instruction set by the second therapy configuration instruction set, the controller is configured, while the second therapy configuration instruction set is being executed, to determine whether the holdoff setting of the second therapy configuration instruction set allows interruption of the second therapy configuration instruction set and, if so, interrupting the second therapy configuration instruction set after completing execution of a portion thereof to execute a portion of the first therapy configuration instruction set.

Additionally or alternatively, if the holdoff settings of the first and second therapy configuration instruction sets allow interruption of each of the first and second therapy configuration instruction sets, the controller is configured to alternate between execution of a portion of the first therapy configuration instruction set and execution of a portion of the second therapy configuration instruction set until completion of all aggregate instruction of one of the first and second therapy configuration instruction sets.

Additionally or alternatively, each aggregate instruction includes an aggregate holdoff setting, and the controller is configured to determine, using aggregate holdoff settings of the aggregate instructions, the portion the first configuration instruction set to execute before switching to execute a portion of the second therapy configuration instruction set.

Additionally or alternatively, each pulse component includes a pulse component holdoff setting, and the controller is configured to determine, using pulse component holdoff settings of the pulse components, the portion the first therapy configuration instruction set to execute before switching to execute a portion of the second therapy configuration instruction set.

Another illustrative and non-limiting example takes the form of an implantable medical device comprising: a housing containing a power source, a controller, and stimulation circuitry; and a lead having a plurality of electrodes thereon, the lead coupled to the housing such that the stimulation circuitry can issue stimulus pulse patterns to a patient via the electrodes; wherein the controller comprises: a memory including steering memory, aggregate memory, pulse memory, and configuration memory; a plurality of pulse definition circuits each including steering logic, aggregate logic, and pulse logic; wherein the steering memory contains steering instruction sets for a plurality of steering programs, each steering program determining which of the electrodes receive a fraction of a total stimulus output amplitude, and the steering logic is configured to implement a selected steering instruction set; wherein the pulse memory contains pulse programs, each having a one or more pulse instructions defining pulse components each having a pulse type and one or more determining characteristics for the pulse type; wherein the aggregate memory contains aggregate instructions for a plurality of aggregated outputs, each pairing a selected steering instruction set with a selected pulse program and defining a number of repetitions, each aggregate instruction including an aggregate holdoff setting, further wherein the aggregate holdoff setting for each aggregate instruction determines whether the aggregate instruction can be interrupted by another therapy configuration; and wherein the configuration memory defines a plurality of therapy configuration instruction sets each having a defined total stimulus output amplitude, and identifying a set of aggregate instructions to be executed for each therapy configuration instruction set; wherein the controller is configured to execute the plurality of therapy configuration instruction sets to generate output pulses using the stimulation circuitry in accordance with instructions from the therapy configuration instruction sets by: initiating execution of a first therapy configuration instruction set; while executing the first therapy configuration instruction set, determining a second therapy configuration instruction set is to be executed; (x) determining whether the aggregate holdoff setting for an ongoing aggregate instruction being executed allows interruption of the ongoing aggregate instruction by the second therapy configuration instruction set and: if so, completing execution of a pulse program of the ongoing aggregate instruction set of the first therapy configuration instruction set and then starting execution of at least a first aggregate instruction set of the second therapy configuration instruction set; or if not, completing execution of the ongoing aggregate instruction set of the first therapy configuration instruction set and then initiating execution of at least the first aggregate instruction of the second therapy configuration to be executed.

Additionally or alternatively, each pulse component includes a pulse component holdoff setting, and the controller is configured to determine, using pulse component holdoff settings of the pulse components, the portion the first therapy configuration instruction set to execute before switching to execute a portion of the second therapy configuration instruction set.

Another illustrative and non-limiting example takes the form of an implantable medical device comprising: a housing containing a power source, a controller, and stimulation circuitry; and a lead having a plurality of electrodes thereon, the lead coupled to the housing such that the stimulation circuitry can issue stimulus pulse patterns to a patient via the electrodes; wherein the controller comprises: a memory including steering memory, aggregate memory, pulse memory, and configuration memory; a plurality of pulse definition circuits each including steering logic, aggregate logic, and pulse logic; wherein the steering memory contains steering instruction sets for a plurality of steering programs, each steering program determining which of the electrodes receive a fraction of a total stimulus output amplitude, and the steering logic is configured to implement a selected steering instruction set; wherein the pulse memory contains pulse programs, each having a one or more pulse instructions defining pulse components each having a pulse type and one or more determining characteristics for the pulse type, each pulse instruction including a pulse holdoff setting; wherein the aggregate memory contains aggregate instruction sets for a plurality of aggregated outputs, each pairing a selected steering instruction set with a selected pulse program and defining a number of repetitions; wherein the configuration memory defines a plurality of therapy configurations each having a defined total stimulus output amplitude and identifying a set of aggregate instructions to be executed for each therapy configuration; further wherein the pulse holdoff setting of each pulse instruction determines whether the pulse instruction allows concurrent execution of another therapy configuration during execution of the pulse instruction; and wherein the controller is configured to execute the plurality of therapy configurations to generate output pulses using the stimulation circuitry in accordance with instructions from the therapy configurations by: initiating execution of a first therapy configuration; while executing the first therapy configuration, receiving a request to start a second therapy configuration; in response to the request to start the second therapy configuration, checking a pulse holdoff setting for a next pulse instruction to be executed in the pulse program and: if the pulse holdoff setting of the next pulse instruction allows concurrent therapy by another therapy configuration, starting the second therapy configuration when starting execution of the next pulse instruction; or else waiting at least until completion of execution of the next pulse instruction before starting execution of the second therapy configuration.

Additionally or alternatively, in response to the request to start the second therapy configuration, the controller is configured to prevent initiation of the second therapy configuration until either: a subsequent pulse instruction has a holdoff setting allowing concurrent therapy by another therapy configuration; or the controller completes execution of at least the pulse program that is executing at the time the request to start the second therapy configuration is received.

Additionally or alternatively, in response to the request to start the second therapy configuration, the controller is configured to prevent initiation of the second therapy configuration until either: a subsequent pulse instruction has a holdoff setting allowing concurrent therapy by another therapy configuration; or the controller completes execution of at least the aggregate instruction set that is executing at the time the request to start the second therapy configuration is received.

Additionally or alternatively, in response to the request to start the second therapy configuration, the controller is configured to prevent initiation of the second therapy configuration until either: a subsequent pulse instruction has a holdoff setting allowing concurrent therapy by another therapy configuration; or the controller completes execution of the first therapy configuration.

Additionally or alternatively, the stimulation circuitry comprises a plurality of digital-to-analog converter circuits including selectable current mirrors, and the total output amplitude is defined in terms of total output current, such that the implantable medical device is configured to deliver current controlled neural stimulation.

Additionally or alternatively, the pulse definition circuit is coupled to the plurality of digital-to-analog converter circuits and is configured to instruct a selected one of the plurality of digital-to-analog converter circuits to use the total output current, and divide the total output current using the selected steering instruction set.

Additionally or alternatively, the stimulation circuitry comprises a plurality of switches configured to control which electrodes receive current from the plurality of digital to analog converter circuits, and the pulse definition circuit is coupled to the plurality of switches and is configured to control the plurality of switches using the selected steering instruction set.

Further examples may include an implantable deep brain stimulation system or a spinal cord stimulation system comprising the implantable medical device of any of the preceding examples, and a clinician programmer adapted to communicate with the implantable medical device and program each of the arbitration mode and holdoff settings stored in the implantable medical device; wherein: for an implantable deep brain stimulation system, the lead is adapted for placement in the brain of a patient; and for a spinal cord stimulation system the lead is adapted for placement in the spinal column of a patient.

Another illustrative and non-limiting example takes the form of an implantable medical device comprising: a housing containing a power source, a controller, and stimulation circuitry; and a lead having a plurality of electrodes thereon, the lead coupled to the housing such that the stimulation circuitry can issue stimulus pulse patterns to a patient via the electrodes; wherein the controller comprises: a memory including steering memory, aggregate memory, pulse memory, and configuration memory; a plurality of pulse definition circuits each including steering logic, aggregate logic, and pulse logic; wherein the steering memory contains steering instruction sets for a plurality of steering programs, each steering program determining which of the electrodes receive a fraction of a total stimulus output, and the steering logic is configured to implement a selected steering instruction set; wherein the pulse memory contains pulse programs, each having a one or more pulse instructions defining pulse components each having a pulse type and one or more determining characteristics for the pulse type; wherein the aggregate memory contains aggregate instructions each defining one or more aggregated outputs, each aggregated output pairing a selected steering instruction set with a selected pulse program and defining a number of repetitions for the selected pulse program to execute with the selected steering instruction set; wherein the configuration memory contains a plurality of therapy configuration instruction sets each having a defined total stimulus output amplitude, an arbitration mode, a holdoff setting, and identifying a one or more aggregate instructions to be executed for each therapy configuration; further wherein the arbitration mode defined in the configuration memory for each therapy configuration instruction set determines whether the therapy configuration instruction sets will wait for completion of portions of other therapy configuration instruction sets before initiating, and the holdoff setting determines whether the therapy configuration instruction set can be interrupted by another therapy configuration instruction set; and wherein the controller is configured to execute the plurality of therapy configurations to generate output pulses using the stimulation circuitry by: initiating execution of a first therapy configuration instruction set; while executing the first therapy configuration instruction set, receiving a request to execute a second therapy configuration instruction set; determining whether the arbitration mode for the second therapy configuration instruction set allows the second therapy configuration instruction set to wait for completion of portions of other therapy configuration instruction sets and, if not, initiating execution of the second therapy configuration instruction set while the first therapy configuration instruction set is executing, or else: determining whether the holdoff setting for the first therapy configuration instruction set allows interruption of the first therapy configuration instruction set by the second therapy configuration instruction set and: if so, completing an ongoing execution of at least a portion of the first therapy configuration instruction set, and then starting execution of the second therapy configuration instruction set; or if not, completing execution of the first therapy configuration instruction set before allowing the second therapy configuration instruction set to be started.

Additionally or alternatively, if the holdoff setting for the first therapy configuration instruction set allows interruption of the first therapy configuration instruction set by the second therapy configuration instruction set, the controller is configured, while the second therapy configuration instruction set is being executed, to determine whether the holdoff setting of the second therapy configuration instruction set allows interruption of the second therapy configuration instruction set and, if so, interrupting the second therapy configuration instruction set after completing execution of a portion thereof to execute a portion of the first therapy configuration instruction set.

Additionally or alternatively, if the holdoff settings of the first and second therapy configuration instruction sets allow interruption of each of the first and second therapy configuration instruction sets, the controller is configured to alternate between execution of a portion of the first therapy configuration instruction set and execution of a portion of the second therapy configuration instruction set until completion of all aggregate instruction of one of the first and second therapy configuration instruction sets.

Additionally or alternatively, each aggregate instruction includes an aggregate holdoff setting, and the controller is configured to determine, using aggregate holdoff settings of the aggregate instructions, the portion the first configuration instruction set to execute before switching to execute a portion of the second therapy configuration instruction set.

Additionally or alternatively, each pulse component includes a pulse component holdoff setting, and the controller is configured to determine, using pulse component holdoff settings of the pulse components, the portion the first therapy configuration instruction set to execute before switching to execute a portion of the second therapy configuration instruction set.

Additionally or alternatively, the stimulation circuitry comprises a plurality of digital-to-analog converter circuits including selectable current mirrors, and the total output amplitude is defined in terms of total output current, such that the implantable medical device is configured to deliver current controlled neural stimulation.

Additionally or alternatively, the pulse definition circuit is coupled to the plurality of digital-to-analog converter circuits and is configured to instruct a selected one of the plurality of digital-to-analog converter circuits to use the total output current, and divide the total output current using the selected steering instruction set.

Additionally or alternatively, the stimulation circuitry comprises a plurality of switches configured to control which electrodes receive current from the plurality of digital to analog converter circuits, and the pulse definition circuit is coupled to the plurality of switches and is configured to control the plurality of switches using the selected steering instruction set.

Additionally or alternatively, the implantable medical device is an implantable deep brain stimulation system comprising the implantable medical device, and a clinician programmer adapted to communicate with the implantable medical device and program each of the arbitration mode and holdoff settings stored in the implantable medical device; wherein the lead is adapted for placement in the brain of a patient.

Additionally or alternatively, the implantable medical device is a spinal cord stimulation system comprising the implantable medical device and a clinician programmer adapted to communicate with the implantable medical device and program each of the arbitration mode and holdoff settings stored in the implantable medical device; wherein the lead is adapted for placement in the spinal column of a patient.

Another illustrative and non-limiting example takes the form of an implantable medical device comprising: a housing containing a power source, a controller, and stimulation circuitry; and a lead having a plurality of electrodes thereon, the lead coupled to the housing such that the stimulation circuitry can issue stimulus pulse patterns to a patient via the electrodes; wherein the controller comprises: a memory including steering memory, aggregate memory, pulse memory, and configuration memory; a plurality of pulse definition circuits each including steering logic, aggregate logic, and pulse logic; wherein the steering memory contains steering instruction sets for a plurality of steering programs, each steering program determining which of the electrodes receive a fraction of a total stimulus output amplitude, and the steering logic is configured to implement a selected steering instruction set; wherein the pulse memory contains pulse programs, each having a one or more pulse instructions defining pulse components each having a pulse type and one or more determining characteristics for the pulse type; wherein the aggregate memory contains aggregate instructions for a plurality of aggregated outputs, each pairing a selected steering instruction set with a selected pulse program and defining a number of repetitions, each aggregate instruction including an aggregate holdoff setting, further wherein the aggregate holdoff setting for each aggregate instruction determines whether the aggregate instruction can be interrupted by another therapy configuration; and wherein the configuration memory defines a plurality of therapy configuration instruction sets each having a defined total stimulus output amplitude, and identifying a set of aggregate instructions to be executed for each therapy configuration instruction set; wherein the controller is configured to execute the plurality of therapy configuration instruction sets to generate output pulses using the stimulation circuitry in accordance with instructions from the therapy configuration instruction sets by: initiating execution of a first therapy configuration instruction set; while executing the first therapy configuration instruction set, determining a second therapy configuration instruction set is to be executed; (x) determining whether the aggregate holdoff setting for an ongoing aggregate instruction being executed allows interruption of the ongoing aggregate instruction by the second therapy configuration instruction set and: if so, completing execution of a pulse program of the ongoing aggregate instruction set of the first therapy configuration instruction set and then starting execution of at least a first aggregate instruction set of the second therapy configuration instruction set; or if not, completing execution of the ongoing aggregate instruction set of the first therapy configuration instruction set and then initiating execution of at least the first aggregate instruction of the second therapy configuration to be executed.

Additionally or alternatively, each pulse component includes a pulse component holdoff setting, and the controller is configured to determine, using pulse component holdoff settings of the pulse components, the portion the first therapy configuration instruction set to execute before switching to execute a portion of the second therapy configuration instruction set.

Another illustrative and non-limiting example takes the form of an implantable medical device comprising: a housing containing a power source, a controller, and stimulation circuitry; and a lead having a plurality of electrodes thereon, the lead coupled to the housing such that the stimulation circuitry can issue stimulus pulse patterns to a patient via the electrodes; wherein the controller comprises: a memory including steering memory, aggregate memory, pulse memory, and configuration memory; a plurality of pulse definition circuits each including steering logic, aggregate logic, and pulse logic; wherein the steering memory contains steering instruction sets for a plurality of steering programs, each steering program determining which of the electrodes receive a fraction of a total stimulus output amplitude, and the steering logic is configured to implement a selected steering instruction set; wherein the pulse memory contains pulse programs, each having a one or more pulse instructions defining pulse components each having a pulse type and one or more determining characteristics for the pulse type, each pulse instruction including a pulse holdoff setting; wherein the aggregate memory contains aggregate instruction sets for a plurality of aggregated outputs, each pairing a selected steering instruction set with a selected pulse program and defining a number of repetitions; wherein the configuration memory defines a plurality of therapy configurations each having a defined total stimulus output amplitude and identifying a set of aggregate instructions to be executed for each therapy configuration; further wherein the pulse holdoff setting of each pulse instruction determines whether the pulse instruction allows concurrent execution of another therapy configuration during execution of the pulse instruction; and wherein the controller is configured to execute the plurality of therapy configurations to generate output pulses using the stimulation circuitry in accordance with instructions from the therapy configurations by: initiating execution of a first therapy configuration; while executing the first therapy configuration, receiving a request to start a second therapy configuration; in response to the request to start the second therapy configuration, checking a pulse holdoff setting for a next pulse instruction to be executed in the pulse program and: if the pulse holdoff setting of the next pulse instruction allows concurrent therapy by another therapy configuration, starting the second therapy configuration when starting execution of the next pulse instruction; or else waiting at least until completion of execution of the next pulse instruction before starting execution of the second therapy configuration.

Additionally or alternatively, in response to the request to start the second therapy configuration, the controller is configured to prevent initiation of the second therapy configuration until either: a subsequent pulse instruction has a holdoff setting allowing concurrent therapy by another therapy configuration; or the controller completes execution of at least the pulse program that is executing at the time the request to start the second therapy configuration is received.

Additionally or alternatively, in response to the request to start the second therapy configuration, the controller is configured to prevent initiation of the second therapy configuration until either: a subsequent pulse instruction has a holdoff setting allowing concurrent therapy by another therapy configuration; or the controller completes execution of at least the aggregate instruction set that is executing at the time the request to start the second therapy configuration is received.

Additionally or alternatively, in response to the request to start the second therapy configuration, the controller is configured to prevent initiation of the second therapy configuration until either: a subsequent pulse instruction has a holdoff setting allowing concurrent therapy by another therapy configuration; or the controller completes execution of the first therapy configuration.

Additionally or alternatively, the stimulation circuitry comprises a plurality of digital-to-analog converter circuits including selectable current mirrors, and the total output amplitude is defined in terms of total output current, such that the implantable medical device is configured to deliver current controlled neural stimulation.

Additionally or alternatively, the pulse definition circuit is coupled to the plurality of digital-to-analog converter circuits and is configured to instruct a selected one of the plurality of digital-to-analog converter circuits to use the total output current, and divide the total output current using the selected steering instruction set.

Additionally or alternatively, the stimulation circuitry comprises a plurality of switches configured to control which electrodes receive current from the plurality of digital to analog converter circuits, and the pulse definition circuit is coupled to the plurality of switches and is configured to control the plurality of switches using the selected steering instruction set.

In some examples implantable medical device may be part of an implantable deep brain stimulation system comprising the implantable medical device, and a clinician programmer adapted to communicate with the implantable medical device and program each of the arbitration mode and holdoff settings stored in the implantable medical device; wherein the lead is adapted for placement in the brain of a patient.

In some examples, the implantable medical device may be part of a spinal cord stimulation system comprising the implantable medical device, and a clinician programmer adapted to communicate with the implantable medical device and program each of the arbitration mode and holdoff settings stored in the implantable medical device; wherein the lead is adapted for placement in the spinal column of a patient.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative stimulation system;

FIG. 2 shows additional details of a pulse generator;

FIG. 3 shows an illustrative prior art arbitration scheme;

FIG. 4 illustrate components or functional blocks of a stimulation control circuit;

FIG. 5 illustrates a steering program;

FIG. 6 illustrates a plurality of pulse phase types and an illustrative pulse program;

FIG. 7 shows illustrative aggregate instructions;

FIG. 8 shows illustrative therapy configurations with arbitration;

FIG. 9 shows an illustrative arbitration logic example;

FIG. 10 shows an example with arbitration defined at the pulse phase level;

FIG. 11 shows an example with arbitration control at the pulse program level;

FIG. 12 shows an example with arbitration control included at the aggregate instruction level; and

FIG. 13A-13D show several examples with arbitration control at therapy configuration and aggregate instruction levels.

### DETAILED DESCRIPTION

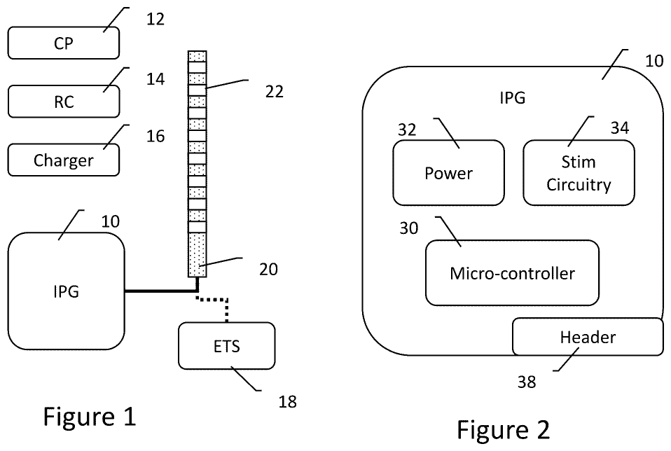

FIG. 1 shows an illustrative stimulation system. An implantable pulse generator (IPG) 10 is operably linked to a lead 20 having electrodes 22. The lead 20 may be placed in any suitable position, such as locations for use in deep brain stimulation (DBS), spinal cord stimulation (SCS) and/or other therapy applied to any of vagus nerve, occipital nerve, sacral nerve, digestive/excretory tracts, etc.

For DBS, the IPG 10 may be positioned, for example, in the upper chest of a patient, with the lead 20, possibly including a lead extension, extending beneath the skin to the head of the patient, where a bore hole through the skull is prepared and the lead is then passed into the brain near a target structure, such as the thalamus, subthalamic nucleus, globus pallidus, or other structures. A lead used in DBS may include a combination of segmented and ring electrodes 22, if desired, such as disclosed in U.S. Pat. Nos. 8,483,237 and 8,321,025, the disclosures of which are incorporated herein by reference.

For SCS, the IPG may be positioned, without limitation, in the region of the buttocks, with the lead extending toward the thoracic spine, for example, such that one or more leads 20 are positioned therein; while a cylindrical lead 20 with a series of ring electrodes 22 is shown in FIG. 1, a paddle lead may be used instead for SCS, having columns of electrodes positioned side-by-side. In SCS, the lead will be extended into the vicinity of the spinal column, such as disclosed variously in any of U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference.

Placement would be intended to bring the electrodes 22 into proximity to target tissue such as neural targets. The IPG 10 can include communication circuitry using, for example and without limitation, inductive, conducted, optical, Bluetooth, Medradio, or other communications modes, frequencies and standards, to communicate while implanted to one or more of a clinician programmer (CP) 12, and a patient remote control (RC) 14. The CP 12 can be used by or at the direction of a physician to select various therapy parameters as is known in the art. The CP 12 may be used to set the various arbitration parameters and controls discussed below. The RC 14 can be used by the patient, typically, to turn therapy on and/or off, to interrogate the IPG 10 to determine device status, and sometimes to adjust therapy settings, such as by changing amplitude of stimulation, or provide patient feedback such as by answering a patient questionnaire.

A charger is shown at 16, and may be used to provide power to the IPG 10 for recharging the batteries of the IPG. Power may be, for example and without limitation, transmitted by inductive coupling between the charger 16 and IPG 10. The present invention is not limited to rechargeable IPGs 10, and may be used as well with non-rechargeable or “primary cell” IPGs 10, in which case the charger 16 may be omitted. An external test system (ETS) 18 is shown. The ETS 18 can be programmed similar to the IPG 10, using, for example, the CP 12, and controlled with RC 14, if desired. The ETS 18 may be used to test therapy programs for efficacy on the patient after the lead 20 has been implanted and before implantation of the permanent IPG 10, as is well known in the art. The circuits and methods discussed below can be used in an ETS 18 as well as an IPG 10.

The plurality of electrodes 22 may be used to deliver targeted therapy in various ways. For example, with a current-controlled therapy, a total quantity of current to be issued via the electrodes can be fractionalized or divided amongst the electrodes to create a volume of activation for the therapy and/or define a central point of stimulation, using known methods. This may be referred to as current steering. For current steering, a system may have a plurality of independent current-controlled outputs, sometimes referred to as multiple independent current control. Voltage-controlled therapy may be issued instead, using a plurality of independent voltage outputs, also allowing therapy to be issued with some degree of control over where the therapy is targeted by selective use of the electrodes. While much of the following is discussed in the context of current-controlled stimulation, arbitration concepts as discussed herein can apply to either current-controlled or voltage-controlled therapy.

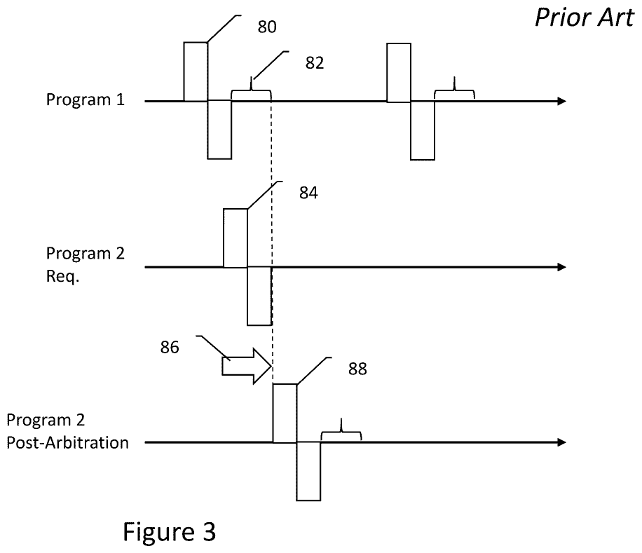

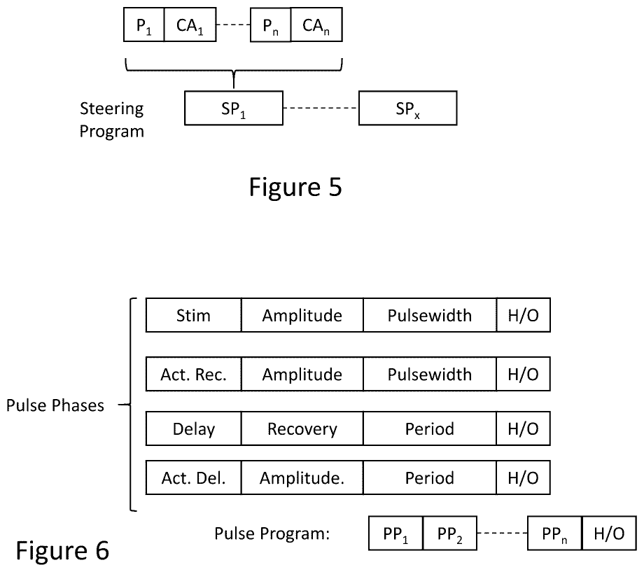

FIG. 2 shows additional details of a pulse generator. The IPG 10 may include separate circuits, sometimes referred to as operational circuitry, including a microcontroller 30 (which may also be implemented as part of a microprocessor if desired), which controls operations of the IPG at a high level. The IPG can include a power source 32, typically a battery (rechargeable or primary cell, as desired), though some systems may be adapted to operate without a battery by receiving power inductively or through other link (such as radiofrequency) and issuing therapy using the received power without long-term storage. A block of stimulation circuitry 34 is also provided, and may include componentry and/or functional blocks as discussed below in relation to FIG. 4. At a high level the stimulation circuitry 34 may include a plurality of current sources and current sinks (for a current-controlled system; a plurality of voltage sources may be used in voltage-controlled systems instead), and control circuitry including for example one or more analog ASICS, as well as switch arrays that implement steering instructions and/or electrode selections. U.S. Pat. No. 10,716,932 provides illustrative details for both current and planned future implementations of the stimulation circuitry 34, and is incorporated herein by reference. The IPG 10 will also house several blocks of memory, which may take any suitable form including, for example and without limitation, RAM, ROM and/or Flash memory.

The IPG 10 may include a conductive outer housing that can serve as a return electrode or indifferent electrode during therapy delivery, as desired. A header 38 provides feedthrough circuitry allowing the IPG 10 to couple to a lead 20 (FIG. 1), with separate electrical connections to each of the electrodes 22. The lead 20 (FIG. 1) may further include any other components (sensors, and optical devices, for example), and the header 38 will include electrical connections for such other components as well, as desired. The header 38 and housing provide a hermetic sealed environment for the operational circuitry 30, 32, 34, and associated memory, all of which may be coupled by various interconnections (wires and/or buses) that are omitted from the drawing.

In some examples in the prior art, a plurality of programs can be set for therapy delivery by the IPG 10. Each program may operate according to a schedule and individual program parameters. A program scheduler may determine whether and when stored therapy programs are called for execution, and may be encoded in stored instructions executed by the micro-controller 30 and/or stimulation circuitry 34. When two programs request output of stimulus at once, the system uses arbitration to determine which program will output stimulus first.

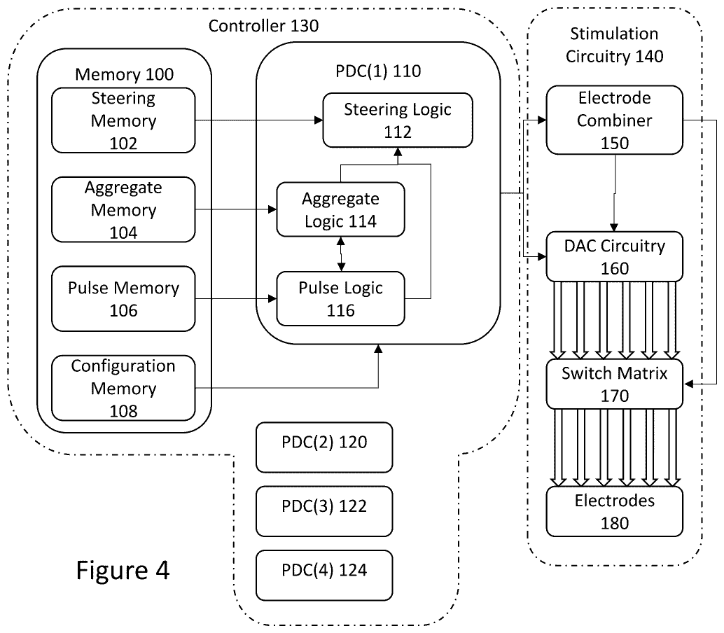

FIG. 3 shows an illustrative prior art arbitration scheme. Here, Program 1 provides a biphasic square wave output shown at 80, and includes a post-stimulation quiet (or quiescent) period at 82. The prior art arbitration logic enforces a hold-off until completion of the quiet period at 82. In the example, shown, Program 2 requests to use the output circuit to issue a therapy pulse as shown at 84. However, this request is denied because Program 1 is still using the output circuitry. The arbitration logic enforces a holdoff or delay, as shown at 86, and does not allow Program 2 to issue the therapy pulse until a later time, as shown at 88, following expiration of the hold-off for the quiet period 82. This arbitration logic prevents Program 2 from operating as intended, but defends the output circuitry from competing commands. With newer circuitry and more sophisticated therapy patterns/programs being studied, a more flexible system is desired.

FIG. 4 illustrates components or functional blocks of a stimulation control circuit. A controller 130 includes a memory 100 and a plurality of pulse definition circuits (PDC) 110, 120, 122, 124. Four PDCs 110, 120, 122, 124 are shown, though any suitable number may be provided. PDC 110 is shown in detail; the other PDCs 120, 122, 124 may be similar or identical.

PDC 110 includes steering logic 112, aggregate logic 114, and pulse logic 116, each interacting with memory 100. Pulse Logic 116 is configured for determining characteristics of each phase of the pulses to be delivered by the system, and references blocks in a pulse memory 106 where selected parameters for particular pulse phases are stored. Steering logic 112 is configured for determining electrode utilization for pulses to be delivered by the system, and references blocks in a steering memory 102 where selected parameters for particular steering modes are stored. Aggregate logic 114 is configured to obtain pairings of pulses and electrode utilization, and references aggregate memory 104 where selected parameters and definitions of particular combinations are stored. An aggregate logic instruction will instruct the pulse logic 116 which portions of the pulse memory 106 to access for defining output pulses, while also instructing the steering logic 112 which portions of the steering memory to use for obtaining electrode utilization instructions. The aggregate logic 114 determines as well the sequence and repetition of output pulses to be used. The PDC 110 will determine from configuration memory 108 which aggregate instructions are to be used by the aggregate logic 114.

In operation, at a particular time, PDC 110 will be enabled and receives a command to execute a portion of the instructions stored in the configuration memory 108, with identification of the relevant addresses of the configuration memory 108 to execute. The command received by the PDC may originate from the scheduler, which, as noted above, may be part of the microcontroller and/or a separate part of the stimulation circuitry. The addresses of the configuration memory 108 determine which portions of the aggregate memory are to be executed, while also carrying additional information. The aggregate logic retrieves the identified portions of aggregate memory in the order prescribed in the configuration memory, and uses the retrieved aggregate instructions to instruct the steering logic and pulse logic to obtain instructions for steering and pulses from identified locations in the steering memory and pulse memory. The present invention in several examples illustrates how arbitration instructions can be made as part of the pulse memory, aggregate memory, and configuration memory, as detailed further below.

The PDC 110 issues control signals to the electrode combiner 150, which in turn provides control signals to the DAC circuitry 160 and switch matrix 170. The DAC circuitry 160 includes a plurality of current mirrors, referred to as “branches”, the quantity of which determines the resolution of the output signal. Any number of current mirrors can be used. As an illustrative example, the DAC circuitry could be configured with 100 branches, each providing 1% of the total output current, thereby providing 1% resolution relative to the maximum current. The electrode combiner 150 determines how the branches of the DAC circuitry 160 will be combined together for each active electrode of the device, and then instructs the switch matrix 170 which switches are to be opened or closed for allowing the combined branches to be output to the electrodes of the device and/or to which electrodes 180 will be grounded or open-circuited during stimulus output.

The functional blocks shown in FIG. 4 may be implemented in several ways. The PDC, for example, can take the form of an application specific integrated circuit (ASIC). For example, there may be a single ASIC comprising all the PDC blocks, or a separate ASIC for each PDC. The ASIC(s) may include a mixed mode integrated circuit carrying and processing both analog and digital signals. Further hardware illustrations and explanations can be found as well in U.S. Pat. No. 10,716,932, the disclosure of which is incorporated herein by reference.

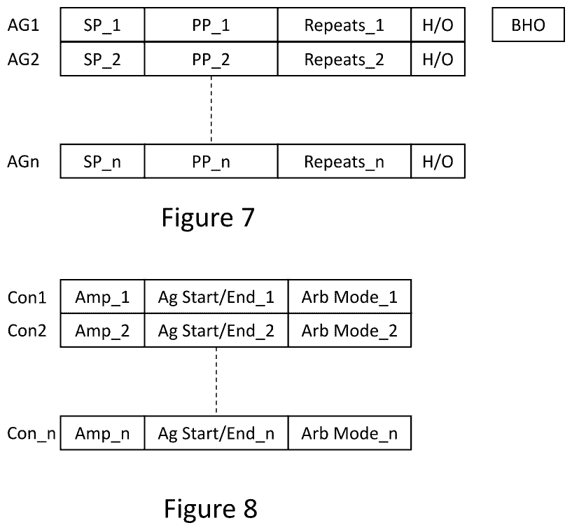

FIG. 5 illustrates a steering program. The memory may hold any desired number of steering programs, such as 8, 16, 32, 64, etc., which are shown as steering programs SP₁ to SPₓ. Each steering program, as illustrated, has memory blocks (at least) for each of the stimulation delivery electrodes of the system. In the illustration, the memory block for the n^{th} electrode includes a polarity definition, Pₙ, and a current allocation definition CAₙ. The polarity definition determines whether the stimulation delivery electrode is an anode or cathode, and may be a single bit in the memory if desired. Polarity definition may use two bits instead, if polarity is defined as any of anode, cathode, ground, or high impedance (which may mean the electrode is available for sensing or is open circuited). Additional bits may be used as desired. The current allocation may use any suitable amount of memory; if resolution of 1% or more is desired, 7 or 8 bits may be used for current allocation for each electrode; fewer bits may achieve lower resolution. Some examples may use a selectable resolution and the steering program may store current allocation for the highest such resolution, with the system simply ignoring one or more bits to provide lower resolution. However the specifics are determined, the illustration shows how each steering program may be stored in memory.

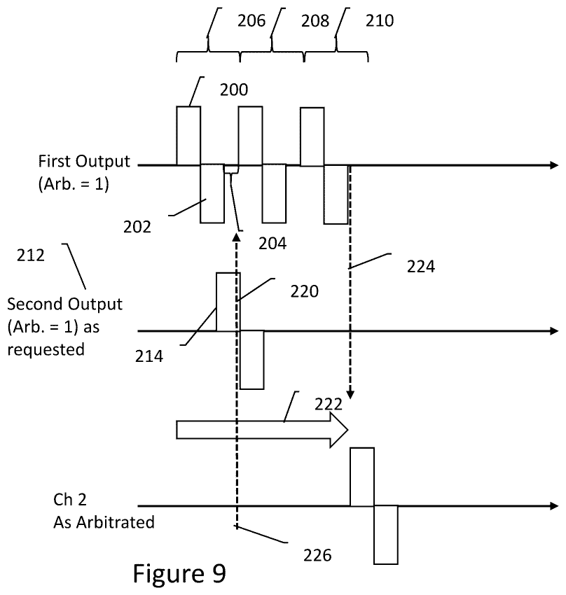

FIG. 6 illustrates a plurality of pulse phase types and an illustrative pulse program. The pulse phases in this illustration have four types: stimulation, active recovery, delay, and active delay. Stimulation phase means that current is being sourced and sunk by the circuitry, using an amplitude defined by the pulse phase and having a duration or pulse width as defined by the pulse phase, and using polarity as defined by a steering program then in use (noting that the aggregate logic defines pairings of pulse program and steering). Active recovery phase also means that current is being sourced and sunk by the circuitry for a duration/pulsewidth, but in this case with a polarity for each electrode being used that is opposite of that defined by a steering program then in use. Delay phase means that no current is actively sourced or sunk by the circuitry for a delay period, and recovery bits are provided that determine whether or not the electrodes of the system are open circuited or grounded, where grounding the electrodes allows passive recovery. During a delay phase the stimulation circuitry can be powered down; an active delay phase is also defined during which no current is actively sourced or sunk, but the stimulation circuitry remains powered on using a specified amplitude during the period defined by the active delay. Active delay phases may be used to prepare for a subsequent stimulation phase.

A pulse program, as shown in the lower part of FIG. 6, defines a sequence of pulse phases PP₁, PP₂ . . . . PPₙ. Any number of pulse phases can be included in a single pulse program, and any number of pulse programs may be stored in the pulse program memory. An enhancement to be further described below is the option to include a holdoff (H/O) setting in each of the pulse phases, as shown.

In some examples, once a pulse program starts, each phase of the pulse program is executed sequentially, without any interruption or holdoff to allow another pulse program or a pulse phase from another program to be executed. This means that a pulse program, once begun, does not stop even if another pulse program or pulse phase of another program is initiated on a different PDC. Alternatively, or additionally, a holdoff setting may be associated with each individual pulse program as a whole, (H/O), as shown, allowing the pulse program to be paused in the middle if another pulse program begins executing and calls for a hold-off.

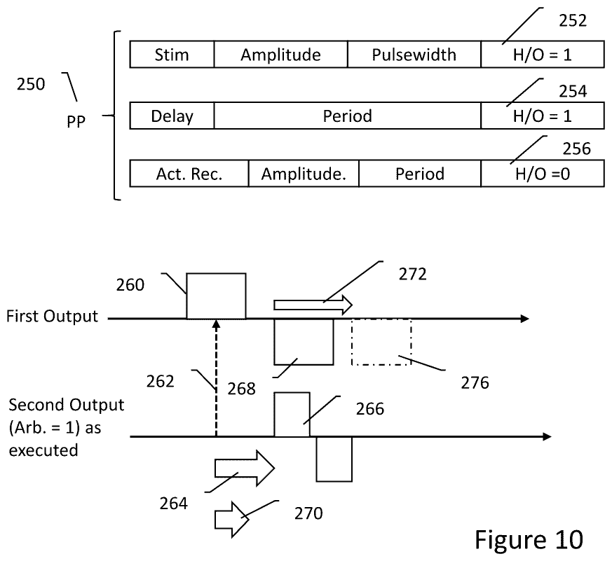

FIG. 7 shows illustrative aggregate instructions. Each aggregate instruction, shown as AG1, AG2 . . . . AGN identifies a steering program (SP_1, SP_2 . . . . SP_n), and a pulse program (PP_1, PP_2 . . . . PP_n), along with a quantity of repeats of the pulse program to be performed in the aggregate instruction. As illustrated, each of the aggregate instructions may include a holdoff setting (H/O), which will be further explained below. In some examples, upon completion of a given aggregate instruction, the PDC executing the aggregate instruction can be paused to allow another PDC (having arbitration on and calling for holdoff) to execute a pulse program, aggregate instruction, or therapy configuration. That is, a series of aggregate instructions may be interrupted once one aggregate instruction is completed, and before a subsequent aggregate instruction begins. In some examples, however, block holdoff (BHO) may be included, optionally, allowing a first aggregate instruction to link to a subsequent aggregate instruction for holdoff purposes, as further explained below.

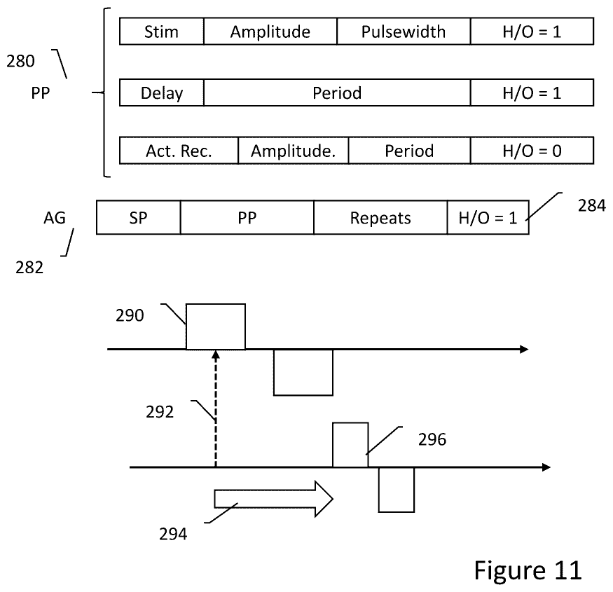

FIG. 8 shows illustrative therapy configurations with arbitration. Each therapy configuration defines an overall amplitude setting (Amp_1, Amp_2 . . . . Amp_n), and defines aggregate instructions to be executed in the therapy configuration. In the example shown, start and end aggregate instructions are identified; in other examples, the aggregate instructions may be identified individually, depending on how the aggregate instruction memory block is organized. In some examples, an arbitration mode can be set at the therapy configuration level.

Reviewing FIGS. 6-8, arbitration settings, holdoffs, or mode definitions may be included at the pulse phase level or pulse program level, as shown in FIG. 6, and/or at the aggregate instruction level, as shown in FIG. 7, and/or at the therapy configuration level as shown in FIG. 8. In some examples, holdoff is set at the lower levels (pulse phase, pulse program, and aggregate instruction), and arbitration enablement is set by the therapy configuration. In other examples, the holdoff and/or arbitration may be omitted at one or more levels. Thus, the illustrations in FIGS. 6-8 may omit holdoff or arbitration in accordance with a particular implementation. Additionally or alternatively, arbitration may be turned on or off globally, if desired. Several examples that follow show how any one of these levels may be used to set arbitration rules for the device.

In these examples, arbitration being enabled means that a given therapy configuration will be operated in accordance with holdoff and arbitration rules applicable to other therapy configurations that also have arbitration enabled. If arbitration is disabled for a therapy configuration, then that therapy configuration will operate without any regard for any other therapy configuration that is in use when the no-arbitration therapy configuration is called or while the no-arbitration therapy configuration is executing. A holdoff means that a given pulse, pulse program, or aggregate instruction, while being executed, prevents another pulse, pulse program, or aggregate instruction from initiating execution on another PDC. Several examples follow.

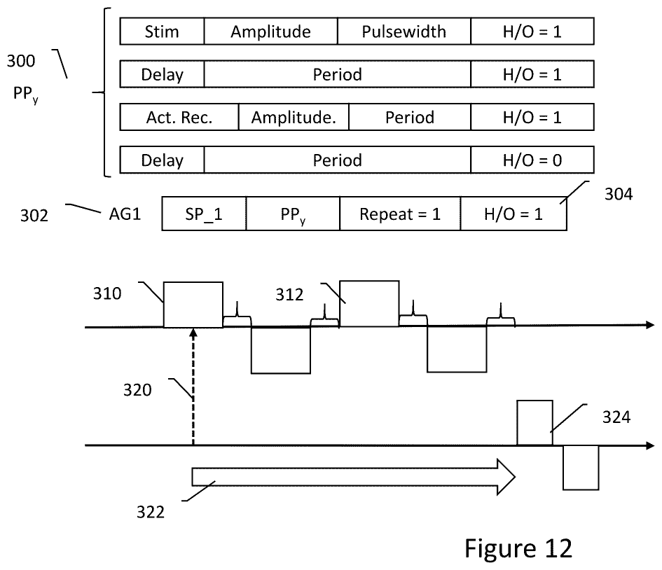

FIG. 9 shows an illustrative arbitration logic example and is also useful for explaining the concepts of a pulse program and aggregate instruction. A first output is generated by the device, with an arbitration setting at the therapy configuration of Arb.=1. The setting of arbitration on for the first output means the output operates with consideration of other outputs in other PDCs. A second output is requested, as illustrated at 212, and the waveform shown at 214 is what the second output would do if Arbitration was off for the second output, or if no other PDC was active at the time the second output is requested. However, as shown at 212, the second output has arbitration on. The request for the first output and the second output may each come from an overall scheduler in the device that determines whether and when each therapy program is requested.

A first output may be defined in several ways with the architecture of FIG. 3. For example, a first output may be defined as a sequence of three phases, 200 (stimulation), 202 (active recovery), and 204 (delay or active delay), stored as a pulse program 206, and the pulse program 206 is called by an aggregate instruction with two repeats. Alternatively, a first output may be defined as a sequence of nine phases (the first three of which are at 200, 202, 204, and the remaining six being apparent from the figure) stored as a pulse program which is executed a single time, without repeats, by an aggregate instruction. In another alternative, three aggregate instructions may each direct execution of the same pulse program, without repeats. In another alternative, each pulse phase may be the single phase of a pulse program, and nine aggregate instructions may be executed in series in accordance with a therapy configuration. In still another alternative, three therapy configuration programs may be executed, with a first of the therapy configurations calling a single aggregate instruction that calls for a pulse program comprising pulse phases 200, 202, 204, and the second and third therapy configurations being the same as the first. As can be seen, with the flexible programming approach in FIG. 3, there are many different ways to define the same pattern of therapy pulses. This flexibility means that, for example, the same pulses could be output to different steering (i.e. if three aggregate instructions are used), or the pulses can be grouped together to ensure consecutive execution (as may be beneficial for burst therapy patterns), or the pulses may be separated from one another by arbitration and hold-off determinations as will be shown below if desired.

Several additional alternative ways to define the first output exist, and the above explanation is not exhaustive. In various examples below, several ways to apply arbitration and holdoffs at the level of pulse phase, pulse program, aggregate instruction, and therapy configuration are further detailed.

While the first output is being executed, with Arb.=1, the entire sequence of 206, 208, 210 would be executed without interruption if these are each part of a single pulse program, in some examples. For such a pulse program, if the holdoff setting is on, then other PDCs executing a therapy configuration having Arb.=1 would have to wait until completion of the sequence 206, 208, 210. In the Figure, another therapy configuration generates a second request, as shown at 220, to issue a therapy output. Due to the arbitration setting and holdoff of the first output, as shown at 222, the second request is subject to a holdoff until completion of the third iteration at 210.

If the first output is generated pursuant to a single aggregate instruction in which each of 206, 208, and 210 are separate iterations of a single pulse program being repeated, with holdoff set to 1 in the aggregate instruction, the result would be the same as if only a single pulse program is present. The first output may instead be generated using three aggregate instructions, allowing different steering configurations for each of the output blocks 206, 208, 210; the pulse program for each aggregate instruction could be the same or different, if desired. A block holdoff can be used to link the aggregate instructions and ensure the outputs occur in a desired succession without interruption. Omitting the block holdoff for three aggregate instructions, however, would allow interruption by the second request at time 226 and execution of the aggregate instructions for output blocks 208 and 210, would then be subject to any arbitration and holdoff settings of the second request.

On the other hand, if the first output is an aggregate instruction and each of 206, 208 and 210 are separate iterations of a single pulse program without a holdoff in the aggregate instruction, the second request would be fulfilled beginning at time 226 instead. That is, as soon as the first iteration of the aggregate program's execution of the pulse program is completed, holdoff enforced by the pulse program 206 would end and the second request could be fulfilled. It can thus be seen that control can be managed based on several rules operating at several levels.

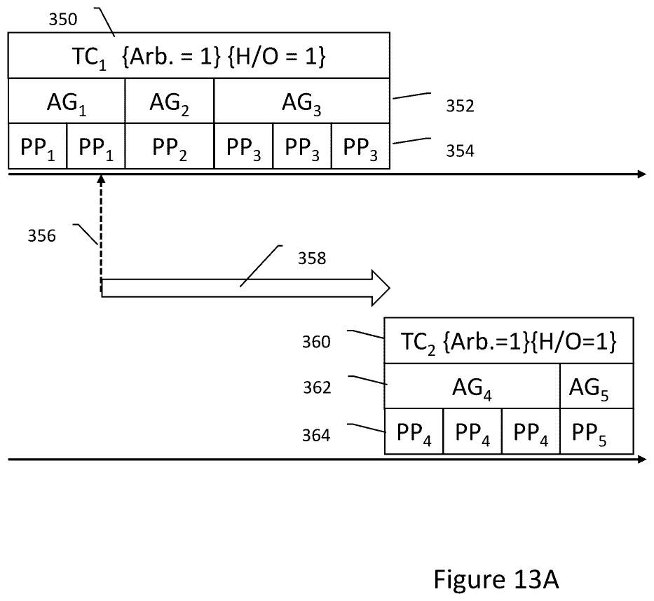

FIG. 10 shows an example with holdoff defined at the pulse phase level. A pulse program is shown at 250, having three phases: a stimulation phase 252, a delay phase 254, and an active recovery phase 256. The active recovery phase 256 has the holdoff set to zero, while the other two phases 252, 254 have the holdoff set to 1, meaning neither may be interrupted by a second therapy configuration or program requesting to issue therapy. In the illustration, as shown at 260, the pulse program 250 has begun execution but is not yet completed when a request from another therapy configuration is received as indicated at 262. Because the arbitration mode for the second output is set to 1, the internal holdoff settings of the first output are next considered. Here, the aggregate instruction has holdoff=0 (not shown), and so the pulse program holdoffs control. The holdoffs for the stimulation pulse 260 and a following delay period are shown at 252, 254, both set to one. This means the request 262 is not fulfilled immediately and must wait. The holdoff setting for the active recovery pulse 268 is set to zero, as indicated at 256, and so the request 262 is allowed, and the pulse at 266 initiates at the same time as the recovery pulse 268 is executed in this example.

In some examples, aggregate instructions are configured to execute the entire pulse program 250 once begun. That would mean that, as shown, each of the recovery pulse 268 of the first output, and the second output, execute simultaneously. In an alternative example, aggregate instructions may instead be configurable to allow interruption of a pulse program in the event that a pulse phase having a holdoff=0 is withheld in the event that a second output having holdoff=1 begins execution; if that were the case, then the active recovery 268 would be delayed as indicated at 272 and executes later, as shown at 276. If pulse program splitting is to be allowed by the aggregate instruction, then the aggregate instruction may have three holdoff settings:
Aggregate instruction Holdoff=0−execute full pulse program once started; holdoff defined by pulse program
Aggregate instruction Holdoff=1−execute full pulse program and repeats, and holdoff any therapy on other PDCs until Aggregate instruction execution completed
Aggregate instruction Holdoff=2−initiate pulse program; holdoff defined by pulse program; and allow interruption of pulse program if another PDC calls for holdoff
   The splitting of pulse programs is optional. Some examples include an additional rule when configuring the pulse program 250. If a holdoff setting of a pulse instruction is set to zero/off, all subsequent pulse instructions in the pulse program would also then be automatically set to zero. Otherwise contrary instructions in the pulse instructions could arise, in which an ongoing pulse in the pulse program at 250 would call for a holdoff while the second output has already started executing.

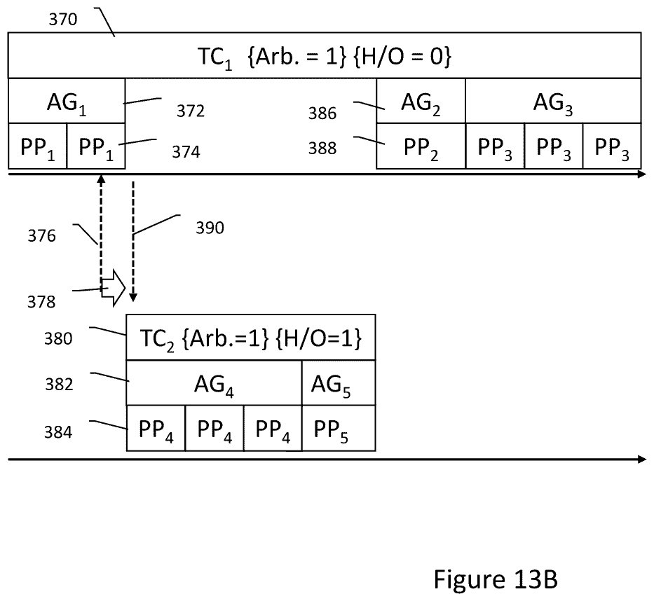

FIG. 11 shows an example with a holdoff implemented at the aggregate instruction level. A pulse program 280 is shown with three phases, stimulation phase, delay phase, and an active recovery phase. The H/O settings are the same as in FIG. 10, with the stimulation and delay phases having holdoff=1, and the active recovery phase having holdoff=0. However, in this case, the aggregate instruction 282 determines each of a steering program, a pulse program, repeats and holdoff setting. The aggregate instruction 282 has holdoff=1, as shown at 284. As a result, the entire execution of the aggregate instruction 282 is subject to holdoff of any other therapy configuration until the aggregate instruction is completed, regardless the holdoff settings in each phase of the pulse program

In the graphic in the lower portion of FIG. 11, the aggregate instruction 282 starts execution of pulse program 280 with pulse 290. While pulse 290 is ongoing, a request 292 comes from another therapy configuration to start a pulse program/phase. Because the aggregate instruction holdoff=1, the entire aggregate instruction, including the entire pulse program, is completed before the request 292 can be fulfilled. As a result, the request 292 is held off as indicated at 294, and is then fulfilled as shown at 296.

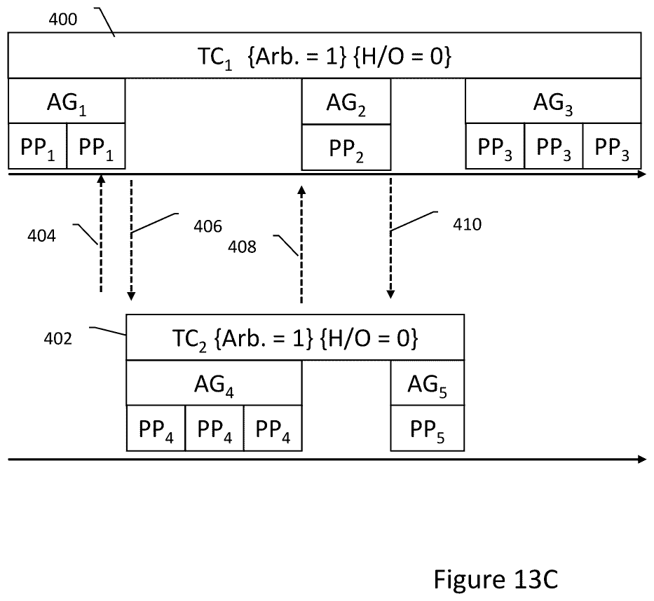

The aggregate instruction 280 in FIG. 11 has no repeats. FIG. 12 shows an example with the holdoff at the aggregate instruction applied to repeats. In FIG. 12, a pulse program, PP_{y} is shown at 300, having four phases (stimulation, delay, active recovery, delay), each with a holdoff setting. An aggregate instruction AG1 is shown at 302, with a steering program SP_1, a pointer to the PP_{y} pulse program, a single repeat, and holdoff=1. The holdoff=1 setting means that during execution of AG1, no other pulse program can interrupt completion of the aggregate instruction.

The graphic at the lower portion of FIG. 11 illustrates how AG1 would be executed. A first execution starts as shown at 310, with a positive pulse, delay, active recovery and delay, before the repeat at 312. During the first execution 310 of AG1, a request to start another pulse program or therapy configuration is received, as indicated at 320. Because AG1 has arbitration mode=1, the request 320 is withheld until completion of AG1, including both the first iteration of pulse program 300 shown at 310, and the repeat or second iteration shown at 312. This delay 322 then allows the requested pulse program execution to occur as shown at 324, after AG1 is completed. It may be noted that the holdoff settings of the pulse phases are not relevant due to the holdoff enforced at the aggregate instruction level in this example.

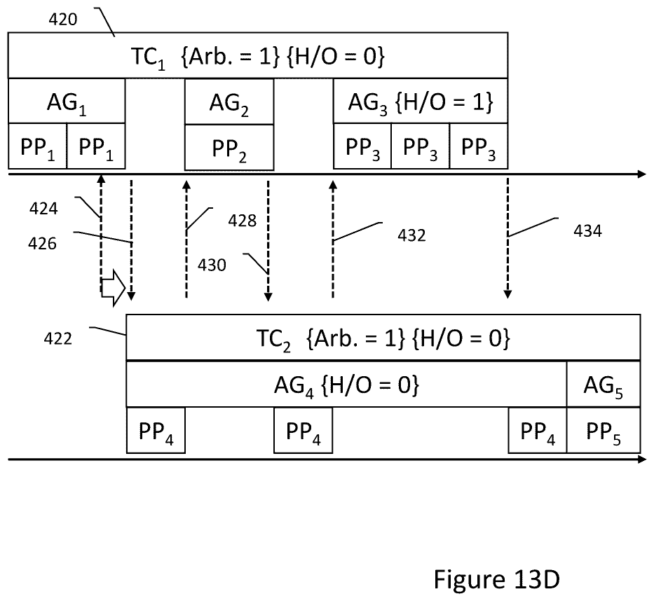

A “burst” is when a series of pulses are delivered at a first pulse repetition rate, followed by a relatively longer pause before a next series of the pulses takes place. Within the neuromodulation space, there is much interest in burst therapy. Structurally, what is shown in FIG. 12 may be very useful to ensure that a burst stimulation, if implemented as an aggregate instruction with repeats, can be executed in its entirety without interruption. In a further illustration, holdoff settings for the aggregate instructions may include more than two values. For example:
Aggregate instruction Holdoff=0−execute full pulse program once started; holdoff defined by pulse program
Aggregate instruction Holdoff=1−execute full pulse program and repeats and holdoff any other arbitration requests until Aggregate instruction execution completed
Aggregate instruction Holdoff=2−initiate pulse program; holdoff defined by pulse program; and allow interruption of pulse program if another PDC calls for holdoff
Aggregate instruction Holdoff=3-execute full pulse program once started; allow interruption between repeats
   Other approaches may be used, as desired. A still higher level of arbitration setting at the therapy configuration level is illustrated in FIGS. 13A-13D.

FIG. 13A shows a therapy configuration 350 configured to execute three aggregate instructions 352, with the first aggregate instruction having one repeat of a first pulse program (steering instructions are not shown but would also be part of each aggregate instruction). The second aggregate instruction has no repeats, and the third aggregate instruction calls for two repeats. During execution of the first aggregate instruction, while the repeat of the pulse program is ongoing, a request 356 is received for initiating execution of a second therapy configuration 360. However, because holdoff=1 for the first therapy configuration 350, the second therapy configuration 360 is subject to holdoff 358 until the first therapy configuration 350 has completed execution.

FIG. 13A can be favorably compared to FIG. 13B. In FIG. 13B, therapy configuration 370 has Holdoff=0, meaning the therapy configuration can be interrupted before completion. A request to execute a second therapy configuration is received at 376, while a first aggregate instruction 372 is being executed, and during a repeat of the pulse program 374. The ongoing pulse program at 374 is then completed (such as by default rule that a pulse program must be allowed to finish, or because an holdoff=1 for the first aggregate instruction 372 or the remaining phases of the ongoing pulse program 374, or because the ongoing pulse program 374 was executing its last phase when the request was received at 376). Because the first therapy configuration 370 has Holdoff=0, interruption is allowed, and second therapy configuration 380 starts. The second therapy configuration is shown with Arbitration=1, meaning it will wait until execution is allowed by the arbitration logic, and Holdoff=1, preventing interruption once started. As a result, the complete second therapy configuration 380, including each aggregate instruction 382 and each pulse program 384 is executed before returning to the first therapy configuration to finish the remaining aggregate instructions 386 and pulse programs thereof.

It may be noted as well that upon the shift of control to the second therapy configuration for execution, the next aggregate instruction in the first therapy configuration becomes the source of a request 390 to interrupt the second therapy configuration. FIG. 13C shows another example where both therapy configurations have Holdoff=0. Though not indicated in the Figure, each aggregate instruction in FIG. 13C has holdoff=1. The first therapy configuration 400 begins execution, and a request to start execution of the second therapy configuration is received at 404. That request 404 is not fulfilled immediately, and instead waits for completion of the first aggregate instruction of the first therapy configuration 400. The second therapy configuration 402 then interrupts execution of the first therapy configuration 400. Due to this interruption, the first therapy configuration issues a request to execute its next aggregate instruction at 406, but is denied until completion of the first aggregate instruction of the second therapy configuration 402, since as shown the aggregate instruction AG4 has holdoff=1, as noted previously.

Because the second therapy configuration has holdoff=0, the system switches back to execute the next aggregate instruction of the first therapy configuration 400 after completing the first aggregate instruction of the second therapy configuration 402. When the second therapy configuration 402 is interrupted, another request is generated at 408 to proceed with the next aggregate instruction of the second therapy configuration 402. That request is not fulfilled until completion of the second aggregate instruction of the first therapy configuration 400. The system thus switches back and forth between the two therapy configurations. For clarity, it may be noted that each of the therapy configurations 400, 402 would be executed here on different PDCs.

Therapy configurations can be broken into parts by allowing switching back and forth without completing aggregate instructions, if desired, as shown in FIG. 13D. In the example of FIG. 13D, each aggregate instruction has holdoff=0, except for AG₃ of the first therapy configuration 420, which is shown with holdoff=1. The result, as shown, is that the aggregate instruction AG₄ of the second therapy configuration executes one pulse program iteration at a time, and waits to repeat the pulse program PP₄ until control returns from the first therapy configuration 420. Therefore, as shown, control shifts back and forth as indicated by the several arrows in the middle of the diagram at 424, 426, 428, 430. When control shifts as shown at 432, however, the AG₃ setting of Arbitration=1 causes each pulse program therein to be executed before the system switches back at 434 to execute the remainder of the second therapy configuration. If the pulse phases of the various pulse programs in FIG. 13D are set with holdoff=0, then the aggregate instruction and pulse program executions can overlap, condensing the therapy still further in time.

Each of these non-limiting examples can stand on its own, or can be combined in various permutations or combinations with one or more of the other examples. The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. An implantable medical device comprising:
a housing containing a power source, a controller, and stimulation circuitry; and
a lead having a plurality of electrodes thereon, the lead coupled to the housing such that the stimulation circuitry can issue stimulus pulse patterns to a patient via the electrodes; wherein the controller comprises:
a memory including steering memory, aggregate memory, pulse memory, and configuration memory;
a plurality of pulse definition circuits each including steering logic, aggregate logic, and pulse logic; wherein:
the steering memory contains steering instruction sets for a plurality of steering programs, each steering program determining which of the electrodes receive a fraction of a total stimulus output, and the steering logic is configured to implement a selected steering instruction set;
the pulse memory contains pulse programs, each having a one or more pulse instructions defining pulse components each having a pulse type and one or more determining characteristics for the pulse type;
the aggregate memory contains aggregate instructions each defining one or more aggregated outputs, each aggregated output pairing a selected steering instruction set with a selected pulse program and defining a number of repetitions for the selected pulse program to execute with the selected steering instruction set;
the configuration memory contains a plurality of therapy configuration instruction sets each having a defined total stimulus output amplitude, an arbitration mode, a holdoff setting, and identifying a one or more aggregate instructions to be executed for each therapy configuration;
the arbitration mode defined in the configuration memory for each therapy configuration instruction set determines whether the therapy configuration instruction sets will wait for completion of portions of other therapy configuration instruction sets before initiating, and the holdoff setting determines whether the therapy configuration instruction set can be interrupted by another therapy configuration instruction set; and
the controller is configured to execute the plurality of therapy configurations to generate output pulses using the stimulation circuitry by:
initiating execution of a first therapy configuration instruction set;
while executing the first therapy configuration instruction set, receiving a request to execute a second therapy configuration instruction set;
determining whether the arbitration mode for the second therapy configuration instruction set allows the second therapy configuration instruction set to wait for completion of portions of other therapy configuration instruction sets and, if not, initiating execution of the second therapy configuration instruction set while the first therapy configuration instruction set is executing, or else:
determining whether the holdoff setting for the first therapy configuration instruction set allows interruption of the first therapy configuration instruction set by the second therapy configuration instruction set and:
if so, completing an ongoing execution of at least a portion of the first therapy configuration instruction set, and then starting execution of the second therapy configuration instruction set; or
if not, completing execution of the first therapy configuration instruction set before allowing the second therapy configuration instruction set to be started.

2. The implantable medical device of claim 1, wherein, if the holdoff setting for the first therapy configuration instruction set allows interruption of the first therapy configuration instruction set by the second therapy configuration instruction set, the controller is configured, while the second therapy configuration instruction set is being executed, to determine whether the holdoff setting of the second therapy configuration instruction set allows interruption of the second therapy configuration instruction set and, if so, interrupting the second therapy configuration instruction set after completing execution of a portion thereof to execute a portion of the first therapy configuration instruction set.

3. The implantable medical device of claim 1, wherein, if the holdoff settings of the first and second therapy configuration instruction sets allow interruption of each of the first and second therapy configuration instruction sets, the controller is configured to alternate between execution of a portion of the first therapy configuration instruction set and execution of a portion of the second therapy configuration instruction set until completion of all aggregate instructions of one of the first and second therapy configuration instruction sets.

4. The implantable medical device of claim 1, wherein each aggregate instruction includes an aggregate holdoff setting, and the controller is configured to determine, using aggregate holdoff settings of the aggregate instructions, the portion the first configuration instruction set to execute before switching to execute a portion of the second therapy configuration instruction set.

5. The implantable medical device of claim 1, wherein each pulse component includes a pulse component holdoff setting, and the controller is configured to determine, using pulse component holdoff settings of the pulse components, the portion the first therapy configuration instruction set to execute before switching to execute a portion of the second therapy configuration instruction set.

6. The implantable medical device of claim 1, wherein the stimulation circuitry comprises a plurality of digital-to-analog converter circuits including selectable current mirrors, and the total output amplitude is defined in terms of total output current, such that the implantable medical device is configured to deliver current controlled neural stimulation.

7. The implantable medical device of claim 6, wherein the pulse definition circuit is coupled to the plurality of digital-to-analog converter circuits and is configured to instruct a selected one of the plurality of digital-to-analog converter circuits to use the total output current, and divide the total output current using the selected steering instruction set.

8. The implantable medical device of claim 6, wherein the stimulation circuitry comprises a plurality of switches configured to control which electrodes receive current from the plurality of digital to analog converter circuits, and the pulse definition circuit is coupled to the plurality of switches and is configured to control the plurality of switches using the selected steering instruction set.

9. An implantable deep brain stimulation system comprising the implantable medical device of claim 1, and a clinician programmer adapted to communicate with the implantable medical device and program each of the arbitration mode and holdoff settings stored in the implantable medical device; wherein the lead is adapted for placement in the brain of a patient.

10. A spinal cord stimulation system comprising the implantable medical device of claim 1, and a clinician programmer adapted to communicate with the implantable medical device and program each of the arbitration mode and holdoff settings stored in the implantable medical device; wherein the lead is adapted for placement in the spinal column of a patient.

11. An implantable medical device comprising:
a housing containing a power source, a controller, and stimulation circuitry; and
a lead having a plurality of electrodes thereon, the lead coupled to the housing such that the stimulation circuitry can issue stimulus pulse patterns to a patient via the electrodes; wherein the controller comprises:
a memory including steering memory, aggregate memory, pulse memory, and configuration memory;
a plurality of pulse definition circuits each including steering logic, aggregate logic, and pulse logic; wherein:
the steering memory contains steering instruction sets for a plurality of steering programs, each steering program determining which of the electrodes receive a fraction of a total stimulus output amplitude, and the steering logic is configured to implement a selected steering instruction set;
the pulse memory contains pulse programs, each having a one or more pulse instructions defining pulse components each having a pulse type and one or more determining characteristics for the pulse type;
the aggregate memory contains aggregate instructions for a plurality of aggregated outputs, each pairing a selected steering instruction set with a selected pulse program and defining a number of repetitions, each aggregate instruction including an aggregate holdoff setting, further wherein the aggregate holdoff setting for each aggregate instruction determines whether the aggregate instruction can be interrupted by another therapy configuration; and
the configuration memory defines a plurality of therapy configuration instruction sets each having a defined total stimulus output amplitude, and identifying a set of aggregate instructions to be executed for each therapy configuration instruction set; and
the controller is configured to execute the plurality of therapy configuration instruction sets to generate output pulses using the stimulation circuitry in accordance with instructions from the therapy configuration instruction sets by:
initiating execution of a first therapy configuration instruction set;
while executing the first therapy configuration instruction set, determining a second therapy configuration instruction set is to be executed;
determining whether the aggregate holdoff setting for an ongoing aggregate instruction being executed allows interruption of the ongoing aggregate instruction by the second therapy configuration instruction set and:
if so, completing execution of a pulse program of the ongoing aggregate instruction set of the first therapy configuration instruction set and then starting execution of at least a first aggregate instruction set of the second therapy configuration instruction set; or
if not, completing execution of the ongoing aggregate instruction set of the first therapy configuration instruction set and then initiating execution of at least the first aggregate instruction of the second therapy configuration to be executed.

12. The implantable device of claim 11, wherein each pulse component includes a pulse component holdoff setting, and the controller is configured to determine, using pulse component holdoff settings of the pulse components, the portion the first therapy configuration instruction set to execute before switching to execute a portion of the second therapy configuration instruction set.

13. An implantable medical device comprising:
a housing containing a power source, a controller, and stimulation circuitry; and
a lead having a plurality of electrodes thereon, the lead coupled to the housing such that the stimulation circuitry can issue stimulus pulse patterns to a patient via the electrodes; wherein the controller comprises:
a memory including steering memory, aggregate memory, pulse memory, and configuration memory;
a plurality of pulse definition circuits each including steering logic, aggregate logic, and pulse logic; wherein:
the steering memory contains steering instruction sets for a plurality of steering programs, each steering program determining which of the electrodes receive a fraction of a total stimulus output amplitude, and the steering logic is configured to implement a selected steering instruction set;
the pulse memory contains pulse programs, each having a one or more pulse instructions defining pulse components each having a pulse type and one or more determining characteristics for the pulse type, each pulse instruction including a pulse holdoff setting;
wherein the aggregate memory contains aggregate instruction sets for a plurality of aggregated outputs, each pairing a selected steering instruction set with a selected pulse program and defining a number of repetitions;
wherein the configuration memory defines a plurality of therapy configurations each having a defined total stimulus output amplitude and identifying a set of aggregate instructions to be executed for each therapy configuration;
further wherein the pulse holdoff setting of each pulse instruction determines whether the pulse instruction allows concurrent execution of another therapy configuration during execution of the pulse instruction; and
wherein the controller is configured to execute the plurality of therapy configurations to generate output pulses using the stimulation circuitry in accordance with instructions from the therapy configurations by:
initiating execution of a first therapy configuration;
while executing the first therapy configuration, receiving a request to start a second therapy configuration;
in response to the request to start the second therapy configuration, checking a pulse holdoff setting for a next pulse instruction to be executed in the pulse program and:
if the pulse holdoff setting of the next pulse instruction allows concurrent therapy by another therapy configuration, starting the second therapy configuration when starting execution of the next pulse instruction; or
else waiting at least until completion of execution of the next pulse instruction before starting execution of the second therapy configuration.

14. The implantable medical device of claim 13, wherein, in response to the request to start the second therapy configuration, the controller is configured to prevent initiation of the second therapy configuration until either:
a subsequent pulse instruction has a holdoff setting allowing concurrent therapy by another therapy configuration; or
the controller completes execution of at least the pulse program that is executing at the time the request to start the second therapy configuration is received.

15. The implantable medical device of claim 13, wherein, in response to the request to start the second therapy configuration, the controller is configured to prevent initiation of the second therapy configuration until either:
a subsequent pulse instruction has a holdoff setting allowing concurrent therapy by another therapy configuration; or
the controller completes execution of at least the aggregate instruction set that is executing at the time the request to start the second therapy configuration is received.

16. The implantable medical device of claim 13, wherein, in response to the request to start the second therapy configuration, the controller is configured to prevent initiation of the second therapy configuration until either:
a subsequent pulse instruction has a holdoff setting allowing concurrent therapy by another therapy configuration; or
the controller completes execution of the first therapy configuration.

17. The implantable medical device of claim 13, wherein the stimulation circuitry comprises a plurality of digital-to-analog converter circuits including selectable current mirrors, and the total output amplitude is defined in terms of total output current, such that the implantable medical device is configured to deliver current controlled neural stimulation.

18. The implantable medical device of claim 17, wherein the pulse definition circuit is coupled to the plurality of digital-to-analog converter circuits and is configured to instruct a selected one of the plurality of digital-to-analog converter circuits to use the total output current, and divide the total output current using the selected steering instruction set.

19. The implantable medical device of claim 17, wherein the stimulation circuitry comprises a plurality of switches configured to control which electrodes receive current from the plurality of digital to analog converter circuits, and the pulse definition circuit is coupled to the plurality of switches and is configured to control the plurality of switches using the selected steering instruction set.

20. An implantable deep brain stimulation system comprising the implantable medical device of claim 13, and a clinician programmer adapted to communicate with the implantable medical device and program each of the arbitration mode and holdoff settings stored in the implantable medical device; wherein the lead is adapted for placement in the brain of a patient.
